(19) United States
(12) Patent Application Publication
(10) Pub. No.: US 2024/0188924 A1
(43) Pub. Date: Jun. 13, 2024

(54) ULTRASOUND ANALYSIS METHOD AND SYSTEM PERFORMING THE SAME

(71) Applicant: STARSHOT PTE. LTD., Singapore (SG)

(72) Inventors: Hao-Li LIU, Taipei City (TW); Chih-Chung HUANG, Tainan City (TW)

(21) Appl. No.: 18/080,434

(22) Filed: Dec. 13, 2022

(51) Int. Cl. A61B 8/00

(52) U.S. Cl. CPC A61B 8/00; A61B 8/52

(57) ABSTRACT

An ultrasound analysis method and a system performing the same are provided. A plurality of ultrasound emission-receiving elements of the system alternately emits a focused ultrasound and a planar ultrasound to a target object. The energy and the focus position of the focused ultrasound are controlled during the emission of the focused ultrasound. The energy level and the direction of travel of the planar ultrasound are controlled during the emission of the planar ultrasound. The system receives scattered echo signals of the planar ultrasound to generate echo mapping signals, thereby reconstructing the echo mapping signals into an ultrasound image to map neurological functional change.

### BACKGROUND OF THE INVENTION

### Field of the Invention

The invention relates to an ultrasound analysis method and a system performing the same, particularly to a method stimulating a target object with a focused ultrasound to obtain ultrasonic images with a planar ultrasound for analysis.

### Description of the Prior Art

Neuroscience is the field of study that studies how the nervous system works, spanning basic science, engineering and medicine. It also has a major impact on the industrialization of pharmaceuticals, biomedicine and medical devices. Thanks to technological advances discovered by novel neuroscience tools, it is believed that great leaps and breakthroughs will be made in the short term to decode and understand human brain function. Governments around the world are also racing to devote significant resources to boosting national brain science research programs. Although most technologies are still in the lab stage, new startups related to neurotechnology have begun to sprout in recent years. Private equity funds and venture capital firms have begun to invest in new start-up companies related to neuroscience, making the basic research and industrial development of neuroscience more popular and becoming an emerging hot research field.

Anatomically, the white matter of the brain is made up of nerve fibers whose function is to connect and transmit nerve impulses. These nerve fibers can be identified by Magnetic Resonance Diffusion Tensor Imaging (MRDTI). Regarding the functional connectivity of the brain, several tools have been developed at present to analyze and map the temporal and spatial activity of the brain. For example, functional magnetic resonance imaging (fMRI) correlates neural activity with the detection of Blood-Oxygen-Level-Dependent (BOLD). It is assumed that neural activities change with the consumption of blood flow. Therefore, when a specific area of the brain is in operation, the blood flow in the specific area changes rapidly and locally. By detecting the running variation of scatter with ultrasound analysis, there is an opportunity to estimate the blood flow changing locally, and then infer that the local nervous function is in action.

For another example, positron emission tomography (PET) is used for monitoring blood metabolism and electrophysiological monitoring; for example, motor evoked potential (MEP), magnetoencephalography (MEG), electroencephalography (EEG) and somatosensory evoked potential (SSEP) have been applied clinically.

In addition, apart from the aforementioned brain activity sensing tools, a brain stimulation device that stimulates the brain from the outside with physical energy is also needed, and can establish a relationship between brain stimulation and perception with the above-mentioned brain perception tools. So far, non-invasive brain stimulation methods include transcranial magnetic stimulation (TMS) and transcranial direct current stimulation (tDCS), which use electromagnetic or electrical energy to induce stimulation to the brain. However, since electromagnetic energy can only be distributed on the brain surface and cannot be precisely focused, stimulation or precise stimulation of subcortical or deeper target brain regions is limited, limiting its ability to explore whole-brain functional connectivity explorations.

Due to many limitations, existing brain stimulation and brain perception tools cannot meet the requirements in the technology of current neuroscience development. In contrast, focused ultrasound provides non-destructive energy levels that can non-invasively and transcranially deliver targeted energy deep into the brain to modulate brain activity. In central nervous system (CNS) applications, use of focused ultrasound includes targeted thermal ablation and targeted blood brain barrier (BBB) opening for the treatment of essential tremor, and more recently the potential of focused ultrasound in modulating neurons have been found.

Using ultrasound to decode neural functions, explore functional brain connectivities or create brain-computer interfaces is new uncharted territory with many niches and advantages, including non-invasive, precise energy delivery and depth, and has great potential as a next-generation brain stimulation tool to explore whole-brain function connectivity.

In terms of mechanism, scientific progress in recent years has found that ultrasound can regulate ion channels on nerve cells and trigger or inhibit initial action potentials, depending on the form of neurons for a given ultrasound exposure parameter. Theoretically, using focused ultrasound to stimulate/inhibit brain activity is a more durable tool. Utilizing the unique strength of focused ultrasound, focused energy can be delivered transcranially deep into the brain, reaching the deepest part of the brain, so theoretically any target location in the brain can be stimulated.

However, at present, stimulation and imaging with the ultrasound still require other brain perception tools to establish the relationship between brain stimulation and perception, just like the aforementioned brain stimulation equipment, so that the cost of establishing the relationship between brain stimulation and perception is high, and it is difficult to confirm the correlation between stimulation and perception in real time. Also, ultrasound is severely occluded by the skull; there is currently no ultrasound technology that can simultaneously process brain stimulation and perception. Therefore, it is necessary to improve the technology for brain perception or brain stimulation with the ultrasound.

### SUMMARY OF THE INVENTION

In view of the problems in the prior art, an objective of the invention is to establish an ultrasound analysis system to perform brain stimulation and brain perception, thereby establishing the relationship between brain stimulation and brain perception. In addition, the invention can use ultrasound to stimulate the brain to enhance brain perception and assist medical personnel to perform treatment. Also, the invention can load the exciter with drugs, and the exciter may be destroyed by ultrasound to release the drug, such that the drug treatment in the brain is observable by utilizing the results of the brain perception in real-time.

According to the objective of the invention, an ultrasound analysis system is provided, which includes an ultrasound transceiver, a concave-to-planar wave converter, a power amplifier, a pulse sequence generator, a mapping unit and a controller. The ultrasound transceiver (a unit combined brain stimulation and perception) is used to emit transcranially a plurality of sets of ultrasound signals to a target object, and receive transcranially a plurality of scattered echo signals respectively generated by the target object, reflecting the plurality of sets of ultrasound signals. The concave-to-planar wave converter connected with the ultrasound transceiver, and used to receive the focused ultrasound of the plurality of sets of ultrasound signals, and convert a concave wave of the focused ultrasound to a plurality of planar ultrasounds. The power amplifier is connected with the concave-to-planar wave converter, and the power amplifier sequentially sends an amplified radio frequency signal via the concave-to-planar wave converter to the ultrasound transceiver according to a pulse signal. The pulse sequence generator is connected with the power amplifier, and the pulse sequence generator generates the pulse signal according to a control signal and transmits the pulse signal to the power amplifier. The mapping unit is connected with the concave-to-planar wave converter, and the mapping unit receives and maps the plurality of scattered echo signals to generate echo mapping signals. The controller is connected with the pulse sequence generator and the mapping unit, and the controller is used to send the control signal to the pulse sequence generator. For an order of each set of the ultrasound signals, the control signal is used to control the ultrasound transceiver to emit the focused ultrasound first and then emit the plurality of planar ultrasounds through the concave-to-planar wave converter. The control signal is used to control an energy level and a focus position of the focused ultrasound while controlling the ultrasound transceiver to emit the focused ultrasound, and the control signal is also used to control an energy level and the direction of travel of the planar ultrasounds while controlling the ultrasound transceiver to emit the planar ultrasounds. The controller is further used to receive the plurality of echo mapping signals and respectively reconstruct a plurality of ultrasound images according to the plurality of echo mapping signals.

In some embodiments, the ultrasound transceiver includes a housing, and a plurality of ultrasound emission-receiving elements. The housing is provided with an inwardly concave surface, and the plurality of ultrasound emission-receiving elements is arranged on the inwardly concave surface. The plurality of ultrasound emission-receiving elements is connected with the power amplifier respectively.

In some embodiments, the plurality of ultrasound emission-receiving elements sends the focused ultrasound and the planar ultrasounds in a multi-channel manner, and the plurality of ultrasound emission-receiving elements changes the energy of the focused ultrasound until a defined indicator appears in one of the plurality of ultrasound images. The defined indicator is used to indicate that a response of the target object to the ultrasound reaches a set threshold. After the defined indicator appears in the ultrasonic image, the ultrasonic emission-receiving elements emit the focused ultrasound with the same energy level as the defined indicator to the target object until the detection and analysis are completed.

In some embodiments, the ultrasound emission-receiving elements respectively emit ultrasounds in different time orders to form a plurality of planar ultrasounds advancing to the target object at different angles.

In some embodiments, the ultrasound emission-receiving elements receive the plurality of scattered echo signals in the multi-channel manner.

In some embodiments, the mapping unit maps the plurality of scattered echo signals to generate the plurality of echo mapping signals based on a velocity variation tracking algorithm.

In some embodiments, the plurality of ultrasound emission-receiving elements is on the inwardly concave surface and arranged in a one-dimensional array, a two-dimensional array, or a concentric ring.

In some embodiments, frequencies of the plurality of ultrasound emission-receiving elements are between 0.1 MHz and 1 MHz.

The number of all ultrasound emission-receiving elements is from 16 to 256. As far as an effective length of the housing is 100 millimeters (mm), the total number of the ultrasound emission-receiving elements is 64. Each of the ultrasound emission-receiving elements is a piezoelectric element, a size of each of the ultrasound emission-receiving elements is 1.6 millimeters (mm), and a distance between each of the ultrasound emission-receiving elements and the ultrasound emission-receiving elements around itself is close to a half-wavelength of the emitted ultrasound frequency. According to another objective of the invention, an ultrasound analysis method is provided, which is applied to an ultrasound analysis system to detect and analyze a target object. The method includes a target object detection stage first and then a target object analysis stage, wherein during the object detection stage, an ultrasound transceiver of the ultrasound analysis system emits transcranially a plurality of ultrasound signals and receives transcranially a plurality of scattered echo signals generated by the target object reflecting the plurality of ultrasound signals, the mapping unit of the ultrasound analysis system calculates to generate a plurality of echo mapping signals according to the plurality of scattered echo signals, and a controller of the ultrasound analysis system reconstructs a plurality of ultrasound images of the target object according to the echo mapping signals. Each set of the ultrasound signals includes a focused ultrasound, and a plurality of ultrasound emission-receiving elements of the ultrasound transceiver emits each set of the ultrasound signals in an order in which first the focused ultrasound is emitted and the focused ultrasound through the concave-to-planar wave converter, and then the plurality of planar ultrasounds is emitted; during the target object detection stage, each time the energy of emitting the focused ultrasound is gradually increased until a defined indicator appears in one of the plurality of ultrasound images, and then the target object analysis stage is completed. Then the method proceeds to the target object analysis stage, during which the process of the ultrasound system is similar to that of the stage of the target object detection stage, but the difference between the two is that the energy level of emitting the focused ultrasound each time during the target object analysis stage is the same as the energy level of the defined indicator appearing in the ultrasound images during the target object detection stage.

Before the target object detection stage, the target object is administered with a plurality of microstructures; during the target object detection stage and the target object analysis stage, the plurality of scattered echo signals is enhanced through an interaction between the plurality of microstructures with the focused ultrasound and an interaction between the plurality of microstructures with the plurality of planar ultrasounds; the controller is further used to reconstruct a moving track of the plurality of microstructures in the plurality of ultrasound images each time.

During the target object analysis stage, the plurality of ultrasound emission-receiving elements of the planar ultrasound is emitted to the target object at different angles, the target object may reflect the plurality of scattered echo signals at different angles, and the controller reconstructs and combines the plurality of scattered echo signals from different angles to form the plurality of ultrasound images.

In summary, in the invention, the focused ultrasound may be applied to the target object in the same ultrasonic system, so that the target object may generate responses of the ultrasound at a focal position of the focused ultrasound, and the ultrasound images are generated when the planar ultrasounds are applied to the target object to observe stimulus responses of the target object to the focused ultrasound; multiple planar ultrasounds at different angles may be emitted to the target object, so that the ultrasound analysis system may generate multiple scattered echo signals at different angles, and further the multiple scattered echo signals from different angles may be used to form clearer ultrasound images of the target object.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a diagram of a structure according to the invention;

FIG. 2 is a timing diagram of a flow of a target object detection stage and a target object analysis stage according to the invention;

FIG. 3 is a diagram of an ultrasound transceiver;

FIG. 4 is another diagram of the ultrasound transceiver;

FIG. 5 is still another diagram of the ultrasound transceiver;

FIG. 6 is a diagram of emitting a planar ultrasound by the ultrasound transceiver;

FIG. 7 is another diagram of emitting the planar ultrasound by the ultrasound transceiver;

FIG. 8 is still another diagram of emitting the planar ultrasound by the ultrasound transceiver;

FIG. 9 is a diagram of a simulated reconstructed ultrasound image of the focused ultrasound;

FIG. 10 is a diagram of a simulated reconstructed ultrasound image of the planar ultrasound;

FIG. 11 is the conventional ultrasound B-mode image;

FIG. 12 is the conventional power Doppler imaging;

FIG. 13 is the high resolution ultrasound image from a flow phantom;

FIG. 14 is a diagram of the ultrasonic image before the focused ultrasound stimulates a skull;

FIG. 15 is a diagram of the ultrasonic image after 1 second of stimulation by the focused ultrasound;

FIG. 16 is a diagram of the ultrasonic image after 10 second of stimulation by the focused ultrasound;

FIG. 17 is a diagram of the ultrasonic image after 100 second of stimulation by the focused ultrasound.

### DESCRIPTION OF THE PREFERRED EMBODIMENTS

Embodiments of the invention will be further explained with the help of the related drawings below. Wherever possible, in the drawings and the description, the same reference numbers refer to the same or similar components. In the drawings, shapes and thicknesses may be exaggerated for simplicity and convenience. It should be understood that the elements not particularly shown in the drawings or described in the specification have forms known to those skilled in the art. Those skilled in the art can make various changes and modifications based on the content of the invention.

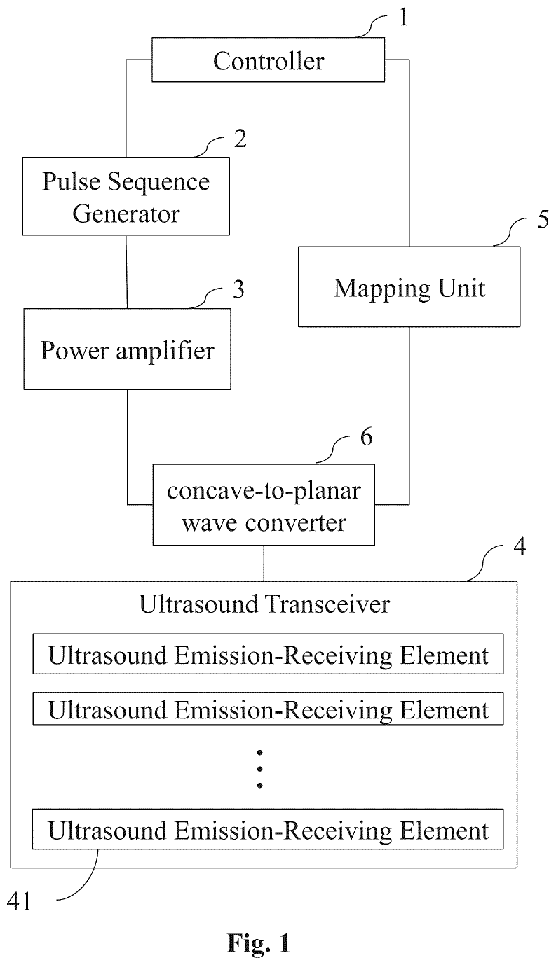

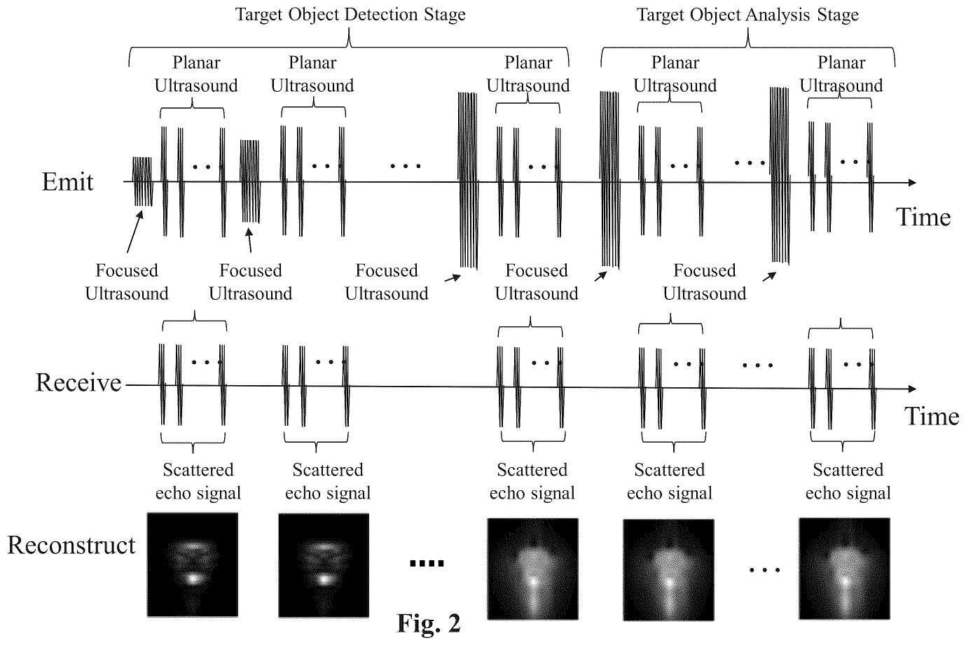

With reference to FIG. 1, the invention is an ultrasound analysis system including a controller 1, a pulse sequence generator 2, a power amplifier 3, an ultrasound transceiver 4, a mapping unit 5, and a concave-to-planar wave converter 6. The controller 1 emits a control signal, wherein the control signal is used to control an order in which a plurality of sets of ultrasound signals is emitted. Each set of the ultrasound signals includes a focused ultrasound. The concave-to-planar wave converter 6 connected with the ultrasound transceiver 4, and used to receive the focused ultrasound of the plurality of sets of ultrasound signals, and convert a concave wave of the focused ultrasound to a plurality of planar ultrasounds. The control signal is used to control an energy level and a focus position of the focused ultrasound while emitting the focused ultrasound and used to control an energy level and a direction of travel of the planar ultrasounds and receive a plurality of echo mapping signals for reconstructing into ultrasound images while emitting the planar ultrasounds. The pulse sequence generator 2 is connected with the controller 1 and used to generate a pulse signal according to a control signal. The power amplifier 3 is connected with both the pulse sequence generator 2 and the concave-to-planar wave converter 6 and sequentially sends an amplified radio frequency signal via the concave-to-planar wave converter 6 to the ultrasound transceiver 4 according to the pulse signal, so that the ultrasound transceiver 4 emits the focused ultrasound or the planar ultrasound to a transcranial target object and receives a plurality of scattered echo signals generated by the transcranial target object reflecting the focused ultrasound or the planar ultrasound (as shown in FIG. 2). The mapping unit 5 is connected with the concave-to-planar wave converter 6 and receives and maps the plurality of scattered echo signals to generate a plurality of echo mapping signals.

In the invention, with reference to FIG. 2, the way for the ultrasound transceiver 4 to emit the focused ultrasound or planar ultrasound to the target object is to emit a plurality of planar ultrasounds immediately after each emission-receiving of the focused ultrasound. The target object may be the skull of a creature, and the object stimulated by the focused ultrasound is the nervous system in the skull. However, the invention is not limited to the above when it is actually implemented, and the target object may also be various organs and tissues of organisms, especially using ultrasound for medical inspection and even for industrial inspection.

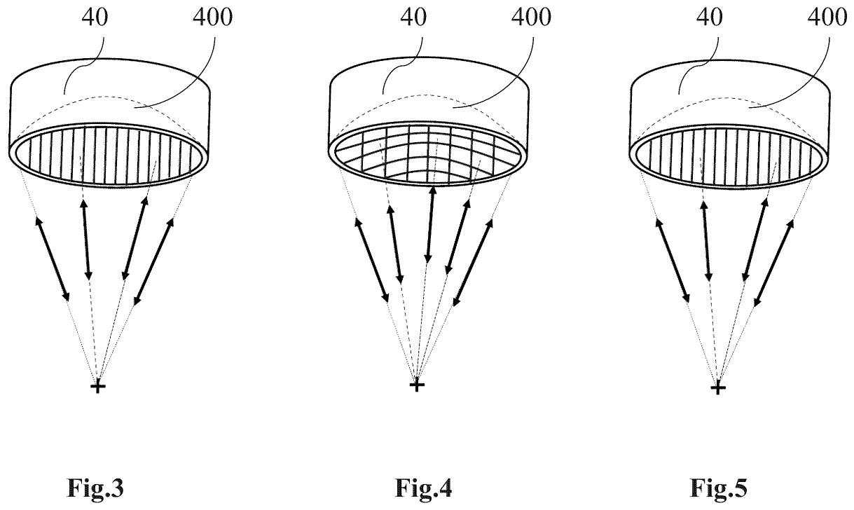

In the invention, with reference to FIGS. 3 to 5, the ultrasound transceiver 4 includes a housing 40, and a plurality of ultrasound emission-receiving elements 41. The plurality of ultrasound emission-receiving elements 41 is arranged on an inwardly concave surface 400 of the housing 40. Further, the plurality of ultrasound emission-receiving elements 41 sends the focused ultrasound and the planar ultrasounds in a multi-channel manner and may change the energy of the focused ultrasound until a defined indicator appears in the ultrasound images. The defined indicator is used to indicate that a response of the target object to the ultrasound reaches a set threshold. The thresholds are different for different target objects; taking the target object as the nervous system in the skull as an example, the threshold is the excitatory response of the nervous system to the ultrasound. Further, when the energy of the focused ultrasound is sufficient to make the defined indicator appear in the ultrasound images, the plurality of ultrasound emission-receiving elements 41 emits the same energy to the target object until the detection and analysis are completed.

Moreover, the ultrasound emission-receiving elements 41 receive the plurality of scattered echo signals in the multi-channel manner. Also, the frequencies of the plurality of ultrasound emission-receiving elements 41 are between 0.1 MHz and 1 MHz.

In the invention, the plurality of ultrasound emission-receiving elements 41 is arranged on the inwardly concave surface 400 in an array or in a concentric ring. As shown in FIG. 3, the plurality of ultrasound emission-receiving elements 41 is arranged in a one-dimensional array on the inwardly concave surface 400; the plurality of ultrasound emission-receiving elements 41 is rectangles with an aspect ratio greater than 1. As shown in FIG. 4, the plurality of ultrasound emission-receiving elements 41 is arranged in a two-dimensional array on the inwardly concave surface 400; the plurality of ultrasound emission-receiving elements 41 is square or circular with an aspect ratio of about 1. As shown in FIG. 5, the plurality of ultrasound emission-receiving elements 41 is arranged in the concentric ring on the inwardly concave surface 400; the plurality of ultrasound emission-receiving elements 41 is circular rings with an aspect ratio greater than 1.

In the invention, the number of all ultrasound emission-receiving elements 41 is from 16 to 256. As far as an effective length of the housing 40 is 100 mm, the total number of the ultrasound emission-receiving elements 41 is 64. Each of the ultrasound emission-receiving elements 41 is a piezoelectric element, a size of each of the ultrasound emission-receiving elements 41 is 1.6 mm, and a distance between each of the ultrasound emission-receiving elements 41 and the ultrasound emission-receiving elements 41 around itself is close to a half-wavelength of the emitted ultrasound frequency.

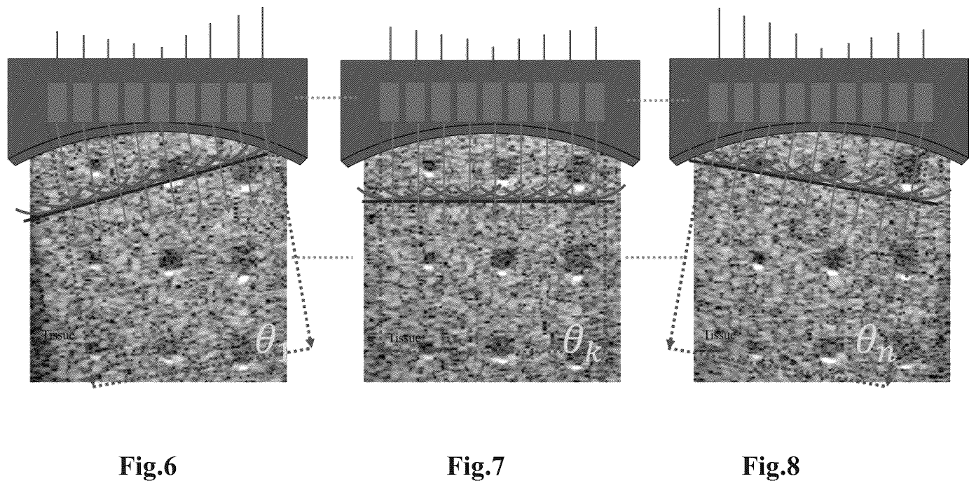

With reference to FIGS. 6 to 8, the plurality of ultrasound emission-receiving elements 41 emits the plurality of planar ultrasounds to the target object at different angles (as θ₁, θₖ, and θₙ respectively shown in FIG. 6, FIG. 7 and FIG. 8) in a multi-channel manner. Further, the ultrasound emission-receiving elements 41 respectively emit the ultrasound in different time sequences to form the plurality of planar ultrasounds advancing to the target object at different angles. In addition, the mapping unit 5 maps the plurality of scattered echo signals to generate the plurality of echo mapping signals respectively based on a velocity variation tracking algorithm.

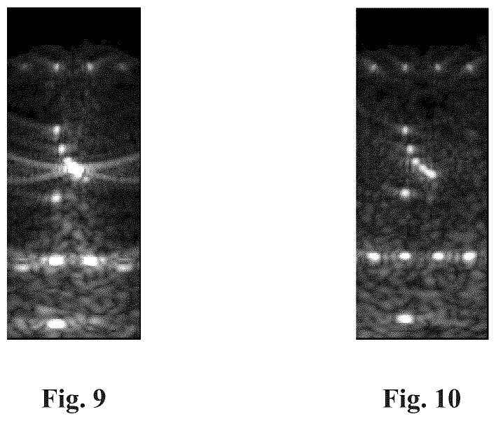

In order to compare the difference in ultrasound imaging of the focused ultrasound and the planar ultrasound, the multi-distance line phantom is used here to be simulated with simulation software respectively to obtain reconstructed images of the simulated multi-distance line phantom. Due to the energy distribution, the image artifacts of the focused ultrasound will be more obvious due to the beam size, so that the ultrasound image of the focused ultrasound is less obvious at the focus position (as shown in FIG. 9). For the planar ultrasound, due to the uniform energy distribution, the image is less affected by artifacts, so the reconstructed ultrasound image of the multi-distance line phantom may be clearly seen (as shown in FIG. 10). Therefore, according to the interferometric synthesis imaging technology and multi-angle deflection imaging, when combined and reconstructed into a synthetic ultrasonic image, the ultrasonic image may obtain finer and more accurate resolution and contrast.

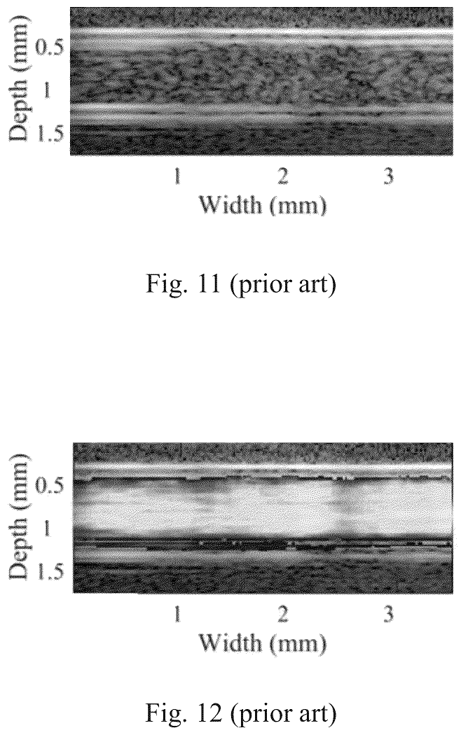

FIG. 11 is the conventional ultrasound B-mode image, and FIG. 12 is the conventional power Doppler imaging.

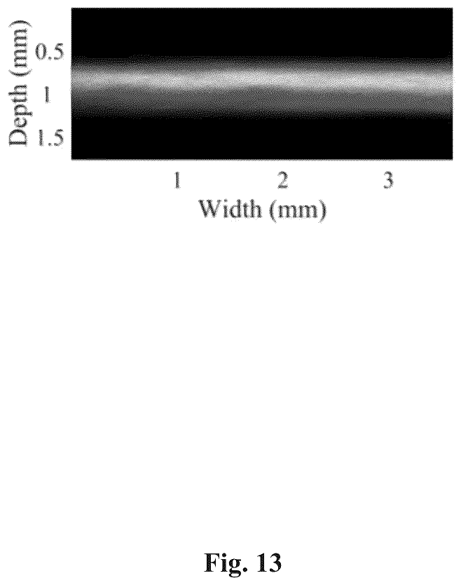

The invention using the super-resolution imaging method, the ultrasonic image is obtained at high speed through the scattered echo signals of the planar ultrasounds, and the ultrasonic images are reconstructed into a super-resolution image by filtering, particle separation, detection, localization, tracking, and mapping, as shown in FIG. 13. FIG. 13 is the high resolution ultrasound image from a flow phantom.

With reference to FIGS. 1 and 2, the invention provides an ultrasound analysis method, which is applied to an ultrasound analysis system to detect and analyze a target object. The method performs a target object detection stage first and then performs a target object analysis stage. During the target object detection stage, the ultrasound transceiver 4 of the ultrasound analysis system emits a plurality of ultrasound signals and receives a plurality of scattered echo signals reflected by the target object; the mapping unit 5 of the ultrasound analysis calculates to generate a plurality of echo mapping signals according to the plurality of scattered echo signals, and the controller of the ultrasound analysis system reconstructs a plurality of ultrasound images of the target object according to the plurality of echo mapping signals. Each set of the ultrasound signals includes a focused ultrasound and a plurality of planar ultrasounds, respectively. For the order of each set of the ultrasound signals, the ultrasound transceiver emits the focused ultrasound first and then the plurality of planar ultrasounds through the concave-to-planar wave converter. During the target object detection stage, each time the energy of emitting the focused ultrasound is gradually increased until a defined indicator appears in one of the plurality of ultrasound images (i.e., the target object analysis stage is completed), and then the method proceeds into the target object analysis stage.

During the target object analysis stage, the action of the ultrasound analysis system is the same as that in the target object detection stage. However, the difference therebetween is that the energy level of emitting the focused ultrasound each time during the target object analysis stage is the same as the energy level of the defined indicator appearing in the ultrasound images during the target object detection stage. The defined indicator is used to indicate that a response of the target object excited by the focused ultrasound reaches a set threshold.

In some embodiments, before the target object detection stage is performed by the ultrasound analysis system, the target object is administered with a plurality of microstructures. During the target object detection stage and the target object analysis stage, the plurality of scattered echo signals is enhanced in intensity through the plurality of microstructures and the focused ultrasounds and interaction between the plurality of microstructures with the plurality of planar ultrasounds. The controller 1 is further used to reconstruct a moving track of the plurality of microstructures in the plurality of ultrasound images each time.

In some embodiments, during the target object analysis stage, the plurality of planar ultrasounds is emitted to the target object at different angles, the target object may reflect the plurality of scattered echo signals at different angles, and the plurality of scattered echo signals from different angles is reconstructed and combined to obtain the plurality of ultrasonic images of the target object formed by the interference of the plurality of scattered echo signals at different angles.

In terms of the principle of observing neural activity with functional magnetic resonance imaging (fMRI), when nerve cells are stimulated by external forces (such as electricity, mechanical force, or light), there are two types of nerve cell activities: facilitation and suppression. In response to the amount of cell activity, the surrounding microvascular bed increase/decrease blood flow and increase/decrease blood oxygen consumption. How the ultrasound analysis system affects nerve cells and how to further observe changes in nerve cell activity are described below.

The designated target position in the brain is stimulated with the focused ultrasound first (i.e., the target object detection stage), and changes in this stage are analyzed through the ultrasound analysis system (i.e., the target object analysis stage).

If the nervous system is stimulated with the focused ultrasound in the intermittent mode under conditions that a burst length of each time using the focused ultrasound is from 0.1ms to 1 ms), the number of shots per second (PRF) is from 100 Hz to 500 Hz, the stimulation is performed for 30 seconds while resting for 30 seconds to form a periodic cycle of at least 300 seconds, and a burst length of the planar ultrasound is from 1ms to 10 ms, which is interspersed between two sections of the stimulation performed by the focused ultrasound; it is observed from continued emission and receiving of the planar ultrasound that the changes in the stimulation of the ultrasound images created by the scattered echo signals of the planar ultrasound tend to increase, which deduces that the parameters of the stimulation performed by the focused ultrasound may stimulate nerve cells, and real-time observation may be performed through the ultrasound system.

Further, if the nervous system is stimulated with the focused ultrasound in another intermittent mode under conditions that the burst length of each time using the focused ultrasound is from 0.1 ms to 1 ms, the number of PRF is 100 Hz to 500 Hz, and the stimulation is performed for at least 300 seconds without resting, and a burst length of the planar ultrasound is from 1 to 10ms, which is interspersed between two sections of the stimulation performed by the focused ultrasound; it is observed from continued emission and receiving of the planar ultrasound that the changes in stimulation of the ultrasound images created by the scattered echo signals of the planar ultrasound tend to decrease, which deduces that the parameters of the stimulation performed by the ultrasound may stimulate nerve cells, and real-time observation may be performed through the device.

In another embodiment of the invention, a plurality of microstructures is added to the target object, and the microstructures are microbubbles, so as to affect the leakage of the microvascular bed for observing the leakage situation.

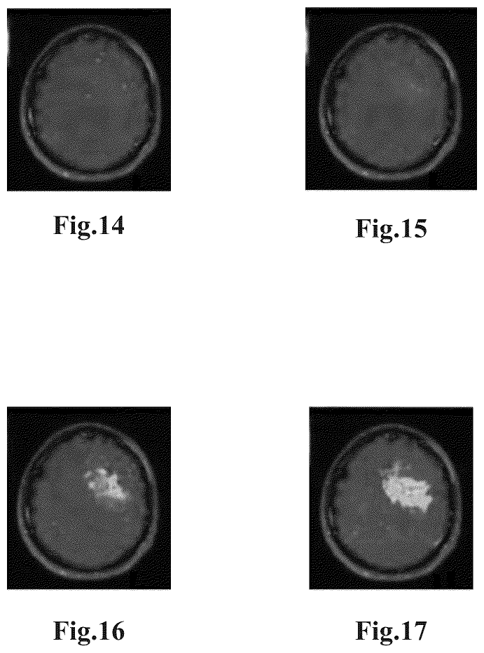

With reference to FIGS. 14 to 17, it can be found in the ultrasound images that the increase of the permeability of the microvascular bed or the blood-brain barrier is effectively promoted; if there is a drug in the microstructure, the drug may be promoted to enter around the focal position of the focused ultrasound for treatment. Further, the microbubbles are intravenously injected into the target object, and the focused ultrasound is used to stimulate the designated position of the target object, an increased change in the permeability of the microvessels is generated; then through the analysis of the ultrasound analysis system, the changes in stimulation of the ultrasound images created by the scattered echo signals of the planar ultrasound are observed. For example, if the burst length using the focused ultrasound is 2-10 ms, the number of shots per second (PRF) is 1-10 Hz, and the stimulation is performed for 30 seconds, it can be observed that the ultrasound images tend to increase. The physiological significance expressed by the changing trend of the ultrasound image at this time: the increase of the permeability of the microvascular bed at the designated position of the focused ultrasound to the target object leads to the increase of the local blood flow.

In summary, the invention stimulates the target object and reconstructs the ultrasound images in the same ultrasound system, so as to observe the stimulus-response of the target object to the focused ultrasound. The planar ultrasounds are emitted to the target object at different angles, so that the ultrasound analysis system may generate multiple scattered echo signals at different angles, and further the multiple scattered echo signals from different angles may be used to generate clearer ultrasound images of the target object.

The above description is only to illustrate the preferred implementation mode of the invention, and is not intended to limit the scope of implementation. All simple replacements and equivalent changes made according to the patent scope of the invention and the content of the patent specification all belong to the scope of the patent application of the invention.

What is claimed is:

1. An ultrasound analysis system, comprising:
an ultrasound transceiver, used to emit transcranially a plurality of sets of ultrasound signals to a target object and receive transcranially a plurality of scattered echo signals respectively generated by the target object reflecting the plurality of sets of ultrasound signals, wherein each set of the ultrasound signals comprises a focused ultrasound;
a concave-to-planar wave converter connected with the ultrasound transceiver, and used to receive the focused ultrasound of the plurality of sets of ultrasound signals, and convert a concave wave of the focused ultrasound to a plurality of planar ultrasounds;
a power amplifier connected with the concave-to-planar wave converter and used to sequentially send an amplified radio frequency signal via the concave-to-planar wave converter to the ultrasound transceiver according to a pulse signal;
a pulse sequence generator connected with the power amplifier and used to generate the pulse signal according to a control signal and transmit the pulse signal to the power amplifier;
a mapping unit connected with the concave-to-planar wave converter and used to receive and map the plurality of scattered echo signals to generate a plurality of echo mapping signals, respectively; and
a controller connected with the pulse sequence generator and the mapping unit and used to send the control signal to the pulse sequence generator, wherein for an order of each set of the ultrasound signals, the control signal is used to control the ultrasound transceiver to emit the focused ultrasound first and then emit the plurality of planar ultrasounds through the concave-to-planar wave converter,
the control signal is used to control an energy level and a focus position of the focused ultrasound while controlling the ultrasound transceiver to emit the focused ultrasound, and the control signal is used to control an energy level and direction of travel of the plurality of the planar ultrasounds while controlling the ultrasound transceiver to emit the plurality of planar ultrasound,
the controller is further used to receive the plurality of echo mapping signals and respectively reconstruct a plurality of ultrasound images according to the plurality of echo mapping signals.

2. The ultrasound analysis system according to claim 1, wherein the mapping unit maps the plurality of scattered echo signals to generate the plurality of echo mapping signals based on a velocity variation tracking algorithm.

3. The ultrasound analysis system according to claim 1, wherein the ultrasound transceiver comprises:
a housing provided with an inwardly concave surface; and
a plurality of ultrasound emission-receiving elements connected with the concave-to-planar wave converter and disposed on the inwardly concave surface.

4. The ultrasound analysis system according to claim 3, wherein the plurality of ultrasound emission-receiving elements sends the focused ultrasound and the plurality of planar ultrasounds in a multi-channel manner, and change energy of the focused ultrasound until a defined indicator appears in one of the plurality of ultrasound images, wherein the defined indicator is used to indicate that a response of the target object to the ultrasound reaches a set threshold.

5. The ultrasound analysis system according to claim 4, wherein the plurality of ultrasound emission-receiving elements respectively emits ultrasounds in different time through the concave-to-planar wave converter orders to form the plurality of planar ultrasounds advancing to the target object at different angles.

6. The ultrasound analysis system according to claim 3, wherein the plurality of ultrasound emission-receiving elements receives the plurality of scattered echo signals in the multi-channel manner.

7. The ultrasound analysis system according to claim 3, wherein the plurality of ultrasound emission-receiving elements is arranged in a one-dimensional array on the inwardly concave surface.

8. The ultrasound analysis system according to claim 3, wherein the plurality of ultrasound emission-receiving elements is arranged in a two-dimensional array on the inwardly concave surface.

9. The ultrasound analysis system according to claim 3, wherein the plurality of ultrasound emission-receiving elements is arranged on the inwardly concave surface in a concentric ring.

10. The ultrasound analysis system according to claim 3, wherein frequencies of the plurality of ultrasound emission-receiving elements are between 0.1 MHz and 1 MHz.

11. An ultrasound analysis method, applied to an ultrasound analysis system to detect and analyze a target object, comprising steps of:
sequentially performing a target object detection stage and a target object analysis stage;
wherein during the target object detection stage and the target object analysis stage, an ultrasound transceiver of the ultrasound analysis system emits transcranially a plurality of sets of ultrasound signals and receives transcranially a plurality of scattered echo signals generated by the target object reflecting the plurality of sets of ultrasound signals, a mapping unit of the ultrasound analysis performs calculation to generate a plurality of echo mapping signals according to the plurality of scattered echo signals, and a controller of the ultrasound analysis system reconstructs a plurality of ultrasound images of the target object according to the plurality of echo mapping signals;
wherein each set of the ultrasound signals comprises a focused ultrasound and a plurality of planar ultrasounds, and for an order of each set of the ultrasound signals, a plurality of ultrasound emission-receiving elements of the ultrasound transceiver emits the focused ultrasound first, and then the plurality of planar ultrasounds through the concave-to-planar wave converter; and
wherein during the target object detection stage, each time the energy of emitting the focused ultrasound is gradually increased until a defined indicator appears in one of the plurality of ultrasound images, and then the target object analysis stage is performed; during the target object analysis stage, an energy level of emitting the focused ultrasound each time is the same as an energy level of the defined indicator appearing in the ultrasound images during the target object detection stage; the defined indicator is used to indicate that a response excited by the focused ultrasound reaches a set threshold.

12. The ultrasound analysis method according to claim 11, wherein the target object is administered with a plurality of microstructures; and
during the target object detection stage and the target object analysis stage, the scattered echo signals are respectively enhanced through an interaction between the plurality of microstructures and the focused ultrasound and an interaction between the plurality of microstructures and the planar ultrasound, and the controller is further used to reconstruct a moving track of the plurality of microstructures in the plurality of ultrasound images.

13. The ultrasound analysis method according to claim 11, wherein during the target object analysis stage, the plurality of ultrasound emission-receiving elements of the ultrasound transceiver transmits the plurality of planar ultrasounds to the target object at different angles, the target object reflect the plurality of scattered echo signals at different angles, and the controller reconstructs and combines the plurality of scattered echo signals from different angles to form the plurality of ultrasound images.
